(19) United States
(12) Patent Application Publication
(10) Pub. No.: US 2011/0177512 A1
(43) Pub. Date: Jul. 21, 2011

(54) METHOD FOR ASSURING AMPLIFICATION OF AN ABNORMAL NUCLEIC ACID IN A SAMPLE

(75) Inventor: Anthony P. Shuber, Mendon, MA (US)

(73) Assignee: Predictive Biosciences, Inc., Lexington, MA (US)

(21) Appl. No.: 12/689,597

(22) Filed: Jan. 19, 2010

(51) Int. Cl. C12Q 1/68; C12P 19/34

(52) U.S. Cl. 435/6.12; 435/91.2

(57) ABSTRACT

The invention generally relates to methods for assuring amplification of an abnormal nucleic acid that is present as a percentage of total nucleic acid in a sample. In certain embodiments, methods of the invention involve providing a sample from a subject, in which the sample includes a total of nucleic acids, in which a percentage of the total are abnormal nucleic acids, extracting the total of nucleic acids from the sample, quantitatively analyzing the extracted nucleic acids, thereby determining an amount of amplifiable nucleic acids in the sample, and providing an amount of the nucleic acids for an amplification reaction that assures amplification of the abnormal nucleic acids in the sample, in which the provided amount is based on results from the quantitatively analyzing step.

### FIELD OF THE INVENTION

The invention generally relates to methods for assuring amplification of an abnormal nucleic acid that is present as a percentage of total nucleic acid in a sample.

### BACKGROUND

Assays have been developed that rely on analyzing nucleic acid molecules from bodily fluids for the presence of mutations, thus leading to early diagnosis of certain diseases such as cancer. In a typical bodily fluid sample however, a majority of the nucleic acid is degraded, and any altered nucleic acids containing mutations of interest are present in small amounts (e.g., less than 1%) relative to a total amount of nucleic acids in the bodily fluid sample. This results in a failure to detect the small amount of abnormal nucleic acid due to stochastic sampling bias.

In order to detect abnormal nucleic acids in the sample, an amplification reaction typically is conducted. However, due to the stochastic nature of the amplification reaction, a population of molecules that is present in a small amount in the sample often is overlooked. In fact, if rare nucleic acid is not amplified in the first few rounds of amplification, it becomes increasingly unlikely that the rare event will ever be detected. Thus, the resulting biased post-amplification nucleic acid population does not represent the true condition of the sample from which it was obtained.

To avoid stochastic sampling, pre-amplification protocols are undertaken to determine the appropriate amount of nucleic acid molecules that need to be provided for the amplification reaction to assure that the abnormal nucleic acids are represented in the post-amplification population. Generally, nucleic acids from bodily fluid are purified and a concentration of total nucleic acids in the sample is determined. The overall concentration of nucleic acids in the sample is used to determine the amount of nucleic acids required to increase the likelihood that the abnormal nucleic acids are represented in the pre-amplification and the post-amplification reaction.

The standard method of determining prospective nucleic acid yield from a sample preparation is to determine a total amount of nucleic acid present in a sample (e.g., based upon optical density measurements). However, that method provides a representation of total nucleic acid and not the amplifiable population that is important for access to small amounts of abnormal nucleic acid.

### SUMMARY

The invention generally relates to methods for assuring amplification of an abnormal nucleic acid from a sample. Methods of the invention go beyond simply determining the total amount of nucleic acid in a sample and instead provide a baseline of usable nucleic acid for amplification. These methods ensure that all nucleic acid populations (e.g., normal and mutated) are represented in assays using amplification to produce usable amounts of nucleic acid product. In essence, methods of the invention enable the detection of small populations of abnormal nucleic acid in a heterogeneous sample without stochastic sampling bias.

Methods of the invention provide a baseline of amplifiable nucleic acid for subsequent amplification reactions. Use of methods of the invention ensures a sufficient population of nucleic acid for analysis of the diversity of nucleic acid species present in a sample. Preferred methods involve quantifying total amplifiable nucleic acid in a sample in order to establish a baseline amount of nucleic acid necessary to insure that a representative amount of an abnormal nucleic acid is present for interrogation. Any known method may be used to quantify amplifiable nucleic acid. However, a preferred method is the polymerase chain reaction (PCR) and, specifically quantitative polymerase chain reaction (QPCR).

Methods of the invention may involve extracting nucleic acids from the sample. Extracting nucleic acid is accomplished by any method known in the art. See for example, Maniatis, et al., Molecular Cloning: A Laboratory Manual, Cold Spring Harbor, N.Y., pp. 280-281 (1982). In certain embodiments, the sample is applied to an affinity column, thereby binding nucleic acids to the column; and then eluting the bound nucleic acid from the column.

In certain embodiments, the sample contains extracellular, circulating nucleic acid molecules. In other embodiments, the nucleic acids are partially degraded. In other embodiments, abnormal nucleic acid is present as about 1% or less of the total amount of nucleic acid molecules in the sample. Generally, the abnormal nucleic acid molecule is indicative of a disease, such as cancer. Exemplary cancers include brain, kidney, liver, adrenal gland, bladder, cervix, breast, stomach, ovaries, esophagus, neck, head, skin, colon, rectum, prostate, pancreas, liver, lung, vagina, thyroid, carcinomas, sarcomas, glioblastomas, multiple myeloma, blood, or gastrointestinal.

Another aspect of the invention provides methods for diagnosing a disease in a subject including analyzing a sample containing an amount of total nucleic acid that enables detection of abnormal nucleic acid without stochastic bias. The method may further include extracting total nucleic acid from a sample, quantitatively analyzing the extracted nucleic acid, and thereby determining an amount of amplifiable nucleic acid molecules in the sample useful for further analysis for the presence of indicia of disease.

### DETAILED DESCRIPTION

The invention generally relates to methods for providing a representative amplifiable population of nucleic acid from a heterogeneous sample. Methods of the invention are applicable to any sample that has a heterogeneous population of nucleic acids.

Methods of the invention are especially useful for detection of abnormal or mutated sequence. Abnormal or mutated nucleic acid is indicative of cancerous or precancerous cells. Without limiting the invention to the detection of any specific type of anomaly, mutations can take many forms, including addition, addition-deletion, deletion, frame-shift, missense, point, reading frame shift, reverse, transition and transversion mutations as well as microsatellite alterations.

In certain embodiments, the abnormal nucleic acid molecule is indicative of a disease, such as cancer. Mutations that are indicative of cancer are known in the art. See for example, Hesketh (The Oncogene Facts Book, Academic Press Limited, 1995).

The sample may be a mammalian sample, e.g. a human tissue or bodily fluid. Certain methods of the invention further involve extracting total nucleic acid from a sample. Generally, nucleic acid is extracted from a biological sample by a variety of techniques such as those described by Maniatis, et al. (Molecular Cloning: A Laboratory Manual, Cold Spring Harbor, N.Y., pp. 280-281, 1982), the contents of which are incorporated by reference herein in their entirety. In certain embodiments, extracting includes introducing the sample to an affinity column, thereby binding the nucleic acid molecules in the sample to the column, and eluting the bound nucleic acid molecules from the column, thereby extracting the nucleic acid molecules from the sample. See, e.g., Abdalla et al. (Application Note 10, DNA Sample Preparation: Isolation of DNA from as Little as 25 μL of Urine Using Norgen's Urine DNA Isolation Kit).

After extraction, the nucleic acid is quantified to determine an amount of amplifiable nucleic acid. Any known method may be used to quantify amplifiable nucleic acid. However, a preferred method is the polymerase chain reaction (PCR) and, specifically quantitative polymerase chain reaction (QPCR). QPCR is a technique based on the polymerase chain reaction, and is used to amplify and simultaneously quantify a targeted nucleic acid molecule. QPCR allows for both detection and quantification (as absolute number of copies or relative amount when normalized to DNA input or additional normalizing genes) of a specific sequence in a DNA sample. The procedure follows the general principle of polymerase chain reaction, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), 2006), Heid et al. (Genome Research 986-994, 1996), Sambrook and Russell (Quantitative PCR, Cold Spring Harbor Protocols, 2006), and Higuchi (U.S. Pat. Nos. 6,171,785 and 5,994,056). The contents of these are incorporated by reference herein in their entirety.

Two common methods of quantification are: (1) use of fluorescent dyes that intercalate with double-stranded DNA, and (2) modified DNA oligonucleotide probes that fluoresce when hybridized with a complementary DNA. In the first method, a DNA-binding dye binds to all double-stranded (ds)DNA in PCR, resulting in fluorescence of the dye. An increase in DNA product during PCR therefore leads to an increase in fluorescence intensity and is measured at each cycle, thus allowing DNA concentrations to be quantified. The reaction is prepared similarly to a standard PCR reaction, with the addition of fluorescent (ds)DNA dye. The reaction is run in a thermocycler, and after each cycle, the levels of fluorescence are measured with a detector; the dye only fluoresces when bound to the (ds)DNA (i.e., the PCR product). With reference to a standard dilution, the (ds)DNA concentration in the PCR can be determined. Like other real-time PCR methods, the values obtained do not have absolute units associated with it. A comparison of a measured DNA/RNA sample to a standard dilution gives a fraction or ratio of the sample relative to the standard, allowing relative comparisons between different tissues or experimental conditions. To ensure accuracy in the quantification, it is important to normalize expression of a target gene to a stably expressed gene. This allows for correction of possible differences in nucleic acid quantity or quality across samples.

The second method uses a sequence-specific RNA or DNA-based probe to quantify only the DNA containing the probe sequence; therefore, use of the reporter probe significantly increases specificity, and allows quantification even in the presence of some non-specific DNA amplification. This allows for multiplexing, i.e., assaying for several genes in the same reaction by using specific probes with differently colored labels, provided that all genes are amplified with similar efficiency.

This method is commonly carried out with a DNA-based probe with a fluorescent reporter (e.g. 6-carboxyfluorescein) at one end and a quencher (e.g., 6-carboxy-tetramethylrhodamine) of fluorescence at the opposite end of the probe. The close proximity of the reporter to the quencher prevents detection of its fluorescence. Breakdown of the probe by the 5′ to 3′ exonuclease activity of a polymerase (e.g., Taq polymerase) breaks the reporter-quencher proximity and thus allows unquenched emission of fluorescence, which can be detected. An increase in the product targeted by the reporter probe at each PCR cycle results in a proportional increase in fluorescence due to breakdown of the probe and release of the reporter. The reaction is prepared similarly to a standard PCR reaction, and the reporter probe is added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a real-time PCR thermocycler, and geometric increase of fluorescence corresponding to exponential increase of the product is used to determine the threshold cycle in each reaction.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Cₜ. Since the quantity of DNA doubles every cycle during the exponential phase, relative amounts of DNA can be calculated, e.g. a sample with a Cₜ of 3 cycles earlier than another has 2³=8 times more template. Amounts of nucleic acid (e.g., RNA or DNA) are then determined by comparing the results to a standard curve produced by a real-time PCR of serial dilutions (e.g. undiluted, 1:4, 1:16, 1:64) of a known amount of nucleic acid.

In certain embodiments, the QPCR reaction involves a dual fluorophore approach that takes advantage of fluorescence resonance energy transfer (FRET), e.g., LIGHTCYCLER hybridization probes, where two oligonucleotide probes anneal to the amplicon (e.g. see U.S. Pat. No. 6,174,670). The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: SCORPIONS probes (e.g., Whitcombe et al., Nature Biotechnology 17:804-807, 1999, and U.S. Pat. No. 6,326,145), Sunrise (or AMPLIFLOUR) primers (e.g., Nazarenko et al., Nuc. Acids Res. 25:2516-2521, 1997, and U.S. Pat. No. 6,117,635), and LUX primers and MOLECULAR BEACONS probes (e.g., Tyagi et al., Nature Biotechnology 14:303-308, 1996 and U.S. Pat. No. 5,989,823).

In other embodiments, the QPCR reaction uses fluorescent Taqman methodology and an instrument capable of measuring fluorescence in real time (e.g., ABI Prism 7700 Sequence Detector). The Taqman reaction uses a hybridization probe labeled with two different fluorescent dyes. One dye is a reporter dye (6-carboxyfluorescein), the other is a quenching dye (6-carboxy-tetramethylrhodamine). When the probe is intact, fluorescent energy transfer occurs and the reporter dye fluorescent emission is absorbed by the quenching dye. During the extension phase of the PCR cycle, the fluorescent hybridization probe is cleaved by the 5′-3′ nucleolytic activity of the DNA polymerase. On cleavage of the probe, the reporter dye emission is no longer transferred efficiently to the quenching dye, resulting in an increase of the reporter dye fluorescent emission spectra.

Methods of the invention go beyond simply determining the total amount of nucleic acid in a sample and instead provide a baseline of usable nucleic acid for amplification. These methods ensure that all nucleic acid populations (e.g., normal and mutated) are represented in assays using amplification to produce usable amounts of nucleic acid product.

The QPCR reaction provide a baseline of amplifiable nucleic acid for subsequent amplification reactions. Methods of the invention go beyond simply determining the total amount of nucleic acid in a sample and instead provide a baseline of usable nucleic acid for amplification. These methods ensure that all nucleic acid populations (e.g., normal and mutated) are represented in assays using amplification to produce usable amounts of nucleic acid product.

Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction, strand displacement amplification and restriction fragments length polymorphism.

Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence.

To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one cycle; there can be numerous cycles) to obtain a high concentration of an amplified segment of a desired target sequence. The length of the amplified segment of the desired target sequence is determined by relative positions of the primers with respect to each other, and therefore, this length is a controllable parameter.

With PCR, it is possible to amplify a single copy of a specific target sequence in genomic DNA to a level that can be detected by several different methodologies (e.g., staining, hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. Amplified target sequences can be used to obtain segments of DNA (e.g., genes) for insertion into recombinant vectors.

Other amplification methods and strategies can also be utilized to detect nucleic acids in biological fluids. For example, another approach would be to combine PCR and the ligase chain reaction (LCR). Since PCR amplifies faster than LCR and requires fewer copies of target DNA to initiate, PCR can be used as first step followed by LCR. The amplified product could then be used in a LCR or ligase detection reaction (LDR) in an allele-specific manner that would indicate if a mutation was present. Another approach is to use LCR or LDR for both amplification and allele-specific discrimination. The later reaction is advantageous in that it results in linear amplification. Thus the amount of amplified product is a reflection of the amount of target DNA in the original specimen and therefore permits quantitation.

LCR utilizes pairs of adjacent oligonucleotides which are complementary to the entire length of the target sequence (Barany F. (1991) PNAS 88:189-193; Barany F. (1991) PCR Methods and Applications 1:5-16). If the target sequence is perfectly complementary to the primers at the junction of these sequences, a DNA ligase will link the adjacent 3′ and 5′ terminal nucleotides forming a combined sequence. If a thermostable DNA ligase is used with thermal cycling, the combined sequence will be sequentially amplified. A single base mismatch at the junction of the oligonoucleotides will preclude ligation and amplification. Thus, the process is allele-specific. Another set of oligonucleotides with 3′ nucleotides specific for the mutant would be used in another reaction to identify the mutant allele. A series of standard conditions could be used to detect all possible mutations at any known site. LCR typically utilizes both strands of genomic DNA as targets for oligonucleotide hybridization with four primers, and the product is increased exponentially by repeated thermal cycling.

A variation of the reaction is the ligase detection reaction (LDR) which utilizes two adjacent oligonucleotides which are complementary to the target DNA and are similarly joined by DNA ligase (Barany F. (1991) PNAS 88:189-193). After multiple thermal cycles the product is amplified in a linear fashion. Thus the amount of the product of LDR reflects the amount of target DNA. Appropriate labeling of the primers allows detection of the amplified product in an allele-specific manner, as well as quantitation of the amount of original target DNA. One advantage of this type of reaction is that it allows quantitation through automation (Nickerson et al. (1990) PNAS 87: 8923-8927).

Methods of the invention may also further involve detecting the abnormal nucleic acid molecules. Detecting may be by any method known in the art. An exemplary method involves using optically labeled probes, e.g., fluorescently labeled probes, that bind to the abnormal nucleic acid molecules, and then detecting the labeled probe bound to the abnormal nucleic acid molecules. Such methods are well known in the art. See, e.g., Lapidus et al. (U.S. Pat. Nos. 5,670,325 and 5,928,870) and Shuber et al. (U.S. Pat. Nos. 6,203,993 and 6,214,558).

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for preparing a heterogeneous sample for amplification of abnormal nucleic, the method comprising:
extracting total nucleic acid from a sample suspected to contain abnormal nucleic acid;
quantitatively analyzing the extracted nucleic acids in order to determine an amount of amplifiable nucleic acids in the sample; and
providing an amount of total nucleic acids for an amplification reaction that assures amplification of the abnormal nucleic acids in the sample.

2. The method according to claim 1, wherein quantitatively analyzing comprises conducting a quantitative polymerase chain reaction (QPCR).

3. The method according to claim 1, wherein the amplification reaction is a polymerase chain reaction.

4. The method according to claim 1, wherein extracting comprises:
introducing the sample to an affinity column, thereby binding the nucleic acids to the column; and
eluting the bound nucleic acids from the column.

5. The method according to claim 1, further comprising conducting the amplification reaction, thereby amplifying the abnormal nucleic acids.

6. The method according to claim 5, further comprising detecting the abnormal nucleic acids.

7. The method according to claim 1, wherein the normal and abnormal nucleic acids are cell-free circulating nucleic acids.

8. The method according to claim 7, wherein the cell-free circulating nucleic acids are partially degraded nucleic acids.

9. The method according to claim 1, wherein the abnormal nucleic acids are present as about 1% or less of the total nucleic acids in the sample.

10. The method according to claim 1, wherein the sample is a tissue or bodily fluid.

11. The method according to claim 10, wherein the bodily fluid is selected from the group consisting of: blood, serum, plasma, urine, spinal fluid, lymphatic fluid, semen, vaginal secretion, ascitic fluid, saliva, mucosa secretion, and peritoneal fluid.

12. The method according to claim 1, wherein the abnormal nucleic acid is indicative of a disease.

13. The method according to claim 12, wherein the disease is a cancer.

14. The method according to claim 13, wherein the cancer is selected from the group consisting of: brain, kidney, liver, adrenal gland, bladder, cervix, breast, stomach, ovaries, esophagus, neck, head, skin, colon, rectum, prostate, pancreas, liver, lung, vagina, thyroid, carcinomas, sarcomas, glioblastomas, multiple myeloma, blood, or gastrointestinal.

15. A method for assuring amplification of cell-free circulating abnormal nucleic acid that are present as a percentage of a total cell-free circulating nucleic acids in a bodily fluid, the method comprising:
providing a bodily fluid from a subject, wherein the fluid comprises a total cell-free circulating nucleic acids, wherein a percentage of the total are abnormal nucleic acids;
extracting the total of cell-free circulating nucleic acids from the fluid;
performing a quantitative polymerase chain reaction on the extracted nucleic acids, thereby determining an amount of amplifiable nucleic acids in the fluid; and
providing an amount of the nucleic acids for a polymerase chain reaction that assures amplification of the abnormal nucleic acids in the fluid, wherein the provided amount is based on results from the quantitative polymerase chain reaction.

16. The method according to claim 15, further comprising conducting the polymerase chain reaction, thereby amplifying the abnormal nucleic acids.

17. The method according to claim 16, wherein the polymerase chain reaction is conducted in the presence of an internal QPCR control.

18. The method according to claim 16, further comprising detecting the abnormal nucleic acids.

19. The method according to claim 15, wherein the abnormal nucleic acids are present as about 1% or less of the total nucleic acid molecules in the fluid.

20. The method according to claim 15, wherein the bodily fluid is selected from the group consisting of: blood, serum, plasma, urine, spinal fluid, lymphatic fluid, semen, vaginal secretion, ascitic fluid, saliva, mucosa secretion, and peritoneal fluid.

21. The method according to claim 15, wherein the total of cell-free circulating nucleic acids comprises partially degraded nucleic acids.

22. A method for diagnosing a disease in a subject, the method comprising:
providing a sample from a subject, wherein the sample comprises a total of nucleic acids, wherein a percentage of the total are abnormal nucleic acids that are indicative of a disease;
extracting the total of nucleic acids from the sample;
quantitatively analyzing the extracted nucleic acids, thereby determining an absolute amount of amplifiable nucleic acids in the sample;
providing an amount of the nucleic acids for an amplification reaction that assures amplification of the abnormal nucleic acids in the sample, wherein the provided amount is based on results from the quantitatively analyzing step;
conducting the amplification reaction; and
detecting the amplified nucleic acids, wherein detection of the abnormal nucleic acids indicates presence of the disease.

23. The method according to claim 22, wherein quantitatively analyzing comprises conducting a quantitative polymerase chain reaction (QPCR).

24. The method according to claim 23, wherein the amplification reaction is a polymerase chain reaction.

25. The method according to claim 22, wherein the normal and abnormal nucleic acids are cell-free circulating nucleic acids.

26. The method according to claim 22, wherein the sample is a tissue or bodily fluid.

27. The method according to claim 22, wherein the bodily fluid is selected from the group consisting of: blood, serum, plasma, urine, spinal fluid, lymphatic fluid, semen, vaginal secretion, ascitic fluid, saliva, mucosa secretion, and peritoneal fluid.

28. The method according to claim 22, wherein the disease is a cancer.

29. The method according to claim 28, wherein the cancer is selected from the group consisting of: brain, kidney, liver, adrenal gland, bladder, cervix, breast, stomach, ovaries, esophagus, neck, head, skin, colon, rectum, prostate, pancreas, liver, lung, vagina, thyroid, carcinomas, sarcomas, glioblastomas, multiple myeloma, blood, or gastrointestinal.
